(19) United States
(12) Patent Application Publication
(10) Pub. No.: US 2013/0296778 A1
(43) Pub. Date: Nov. 7, 2013

(54) Wheel Operated Drug Delivery Device

(71) Applicants: Camilla Damgaard-Soerensen, Lyngby (DK); Lars Eilertsen, Fredensborg (DK); Soeren Kjellerup Hansen, Fjenneslev (DK)

(72) Inventors: Camilla Damgaard-Soerensen, Lyngby (DK); Lars Eilertsen, Fredensborg (DK); Soeren Kjellerup Hansen, Fjenneslev (DK)

(73) Assignee: NOVO NORDISK HEALTHCARE AG, Zurich (CH)

(21) Appl. No.: 13/991,583

(22) PCT Filed: Dec. 6, 2011
(86) PCT No.: PCT/EP2011/071853

§ 371 Date: Jun. 24, 2013

Related U.S. Application Data
(60) Provisional application No. 61/421,236, filed on Dec. 9, 2010.

(30) Foreign Application Priority Data
Dec. 6, 2010 (EP) 10193805.8

(51) Int. Cl. A61M 5/24; A61M 5/315

(52) U.S. Cl. CPC A61M 5/2448; A61M 5/31586
USPC 604/82

(57) ABSTRACT

A drug delivery device (1) for mixing substances and administering a final drug product, the device comprising a container having compartments for individual storage of the substances and means for allowing the substances to be mixed. Pistons are arranged in the container and adapted to be moved by a piston driver which is operatively coupled with a user manipulable wheel (106).

### FIELD OF THE INVENTION

The present invention relates generally to medication delivery devices, and in particular to such devices which are capable of mixing separately stored substances before administration to a subject.

### BACKGROUND OF THE INVENTION

Prefilled syringes containing a specific volume of medication when initially provided by the manufacturer are well known in the art of medical devices. Such syringes may e.g. comprise a reservoir barrel provided with a needle interface at one end and a piston at the other end. For transport and storage reasons the needle interface end is typically closed, e.g. by a tip cap or a penetrable rubber septum. In use, an operator mounts an injection needle on the needle interface end (whereby the rubber septum may be penetrated), inserts the needle at the desired injection site and empties, or partly empties, the syringe by slowly pressing the piston down through the barrel using an appertaining piston rod.

Some types of medication need to be on a specific form in order to ensure an acceptable shelf life. For example, some products for treating bleeding disorders are lyophilised and stored as powders until just prior to administration. Dedicated mixing devices exist offering a fast and easy reconstitution of the lyophilised product using a suitable solvent, such as e.g. water. One such type of mixing device is the so-called dual chamber syringe. A dual chamber syringe conventionally comprises a reservoir barrel with an outlet end and two pistons, one of the pistons (the front piston) providing a separating wall effectively dividing the reservoir into two chambers, and the other piston (the rear piston) providing a seal. During storage the powder product is comprised in one of these chambers and the solvent is comprised in the other chamber. Typically, the solvent is comprised in the rear chamber between the two pistons, such that an operator during use of the device may transfer the solvent to the front chamber around the front piston, e.g. via a bypass channel, by pressing the rear piston towards the front piston. An example of such a dual chamber syringe is e.g. found in U.S. Pat. No. 5,788,670 (Schott Glas).

A common drawback of both single chamber and dual chamber syringes is uneven, or jerky, piston motion due to stick-slip effects arising from the interaction between the piston material and the reservoir wall. This results in the user having less control of the speed with which the piston is advanced and may cause a too fast and uncomfortable drug delivery, a too large volume to be delivered, and/or a too fast transfer of solvent from one chamber to another, leading to an undesired foaming of the final mixed product.

In order to minimise these undesired effects a perfectly axial and concentric driving force must be applied to the piston. For a syringe of the type shown in U.S. Pat. No. 5,788,670 this means that the piston rod must be depressed by a perfectly axial and concentric force from e.g. the users thumb. This can be a very difficult task, especially if the stroke is long and the piston rod is tiltable. In that case it is virtually impossible to operate the piston rod as desired using only one hand. Additionally, oftentimes, and in particular when it comes to treatment of a bleeding disorder, the user may be under a certain time pressure, which adds to the general confusion of the situation and challenges the fine motor skills even more.

U.S. Pat. No. 6,419,656 (Arzneimittel GmbH Apotheker Vetter & Ravensburg) discloses a dual chamber syringe provided with a brake system which allegedly prevents the piston from advancing too rapidly in the reservoir. The brake system comprises a number of lugs distributed along the piston rod which will successively ratchet on tabs on an end cap of the syringe when the piston rod is depressed. However, the lugs are arranged in pairs offset by 90° to the preceding and following pairs, which implies that each time the piston rod has been advanced an increment the user has to twist the piston rod to align the lugs with cutouts in the end cap in order to be able to advance it further. This is impractical in situations where delivery of more than just a minor volume of the contents is desired because it requires both a two handed operation and a constant change in hand positions on the syringe when shifting between axial advancement and twisting of the piston rod.

When administering intravenous drugs it is important to check whether the delivery element, e.g. the infusion needle, is properly positioned in a vein. This is to ensure that the drug is delivered directly to the bloodstream as intended. The check may be done by performing an aspiration once the delivery element has been inserted. For a dual chamber type of device such an aspiration is typically carried out by piston retraction. However, this piston retraction is often cumbersome and requires a change of hand position on the device and/or two handed operation.

WO 2009/095129 (SHL Group AB) discloses an injector comprising a two-part housing, a dual compartment container disposed therein, and a scroll wheel. The scroll wheel is operatively connected to the housing and adapted for operation by a finger of a user for causing a sequential collapse of the two container compartments. By operation of the scroll wheel a front housing part slides axially within a rear housing part, whereby a piston rod is advanced in the container. However, thereby the length of the injector changes during mixing and delivery of the medicament. This may be confusing and awkward to some users.

DE 197 32 909 (Henke-Sass, Wolf GmbH) shows a single compartment syringe device having two user operable drive wheels arranged in parallel for activation of a piston rod adapted for advancement of a piston. The piston rod is arranged between the drive wheels, presumably providing a driving force to a central portion of the piston. However, the dual drive wheel arrangement may be difficult to operate properly because of the distance between the contact surfaces.

### SUMMARY OF THE INVENTION

It is an object of the invention to provide a solution which eliminates, or at least reduces, drawbacks of the prior art.

In particular, it is an object of the invention to provide a drug delivery device which has an improved piston motion control, thereby inter alia minimising the risk of too rapid piston advancement and/or overdosing.

It is a further object of the invention to provide such a device which is intuitive and easy to operate and which has a both compact and ergonomic construction.

It is an even further object of the invention to provide a drug mixing and delivery device where all device-related operations can be performed using only one hand and where the different operations do not require different hand positions.

It is an even further object of the invention to provide a drug mixing and delivery device which is operationally symmetrical in the sense that it can be used equally easily and in the exact same way by right-handed and left-handed people.

It is an even further object of the invention to provide a drug mixing and delivery device which signals to the user when the drug mixing phase is over and the delivery phase begins.

It is an even further object of the invention to provide an operationally robust drug mixing and delivery device which requires relatively few constructional components thereby reducing the manufacturing costs and making the device suitable for being disposed of after use.

In the disclosure of the present invention, aspects and embodiments will be described which will address one or more of the above objects or which will address objects apparent from the below disclosure as well as from the description of exemplary embodiments.

In a drug delivery device embodying the principles of the invention piston movement is controlled by a user manipulable activation element which is rotatable about an axis perpendicular to the general axis of the drug container. Thereby, the user has greater control of the force profile applied to the piston while at the same time a repetitive motion is required to advance, or retract, the piston more than one increment in the container. Notably, this repetitive motion can be performed by a single finger.

In a first aspect of the invention a drug delivery device is provided comprising a container for holding the drug, the container having an outlet and a general axis, a piston capable of movement along the general axis, and a piston driver adapted to cooperate with the piston. The drug delivery device further comprises a handle portion from which at least a portion of an activation member protrudes. The activation member is operatively coupled with the piston driver, to thereby enable user selective movement of the piston in the container, and is arranged rotatably about an axis perpendicular to the general axis.

The particular rotational arrangement of the activation member provides for a one handed operation of the drug delivery device because the user can hold the handle portion in the palm of his/her hand while operating the activation member with one of the fingers, e.g. the thumb or the index finger.

In preferred embodiments the container is arranged stationarily with respect to the handle portion, i.e. the position of the container relative to the handle portion is fixed. This enables the user to be in comfortable control of the device during operation, as the length of the device does not change. In other words, the user needs only concentrate on the manipulation of the activation member and does not have to motorically take account of alterations of the device dimensions.

In that respect, the drug delivery device may further comprise a housing integrated with the handle portion for accommodation of the piston driver and at least a portion of the container.

At least a portion of the housing may comprise a single piece element, or mechanically joined elements, having a fixed axial dimension, so as to define a non-variable axial distance between the handle portion and the container.

Alternatively, a web-like support construction may be provided for fixed arrangement of the container relative to the handle portion.

The activation member may be arranged such that the user is only able to induce a limited angular change to its position upon each operation thereof. This can e.g. be done by providing an opening in the handle portion that allows only a segment of the activation member to be accessible for user operation. Due to the coupling between the activation member and the piston driver a limited angular change to the position of the activation member results in a limited axial displacement of the piston in the container. Thereby it is ensured that the piston cannot be forced to advance, or retract, more than a fraction of the total distance in the container in response to a single operation of the activation member.

The activation member may e.g. be a wheel member, or disc member, rotatably mounted on a shaft in the handle portion. The wheel member may be coupled with the piston driver either directly or via one or more force transmission elements. In the former case, the wheel member may comprise a number of engagement segments distributed on one, e.g. planar, side thereof and adapted to successively engage with respective complementary engagement segments arranged along a portion of the piston driver. This type of coupling requires no additional components and further allows the piston driver to be positioned along a diameter of the wheel member, whereby a symmetrical device construction is enabled. In the latter case, the wheel member may e.g. be coupled with the piston driver via a pinion gear such that a rotational movement of the wheel member is converted to a translational movement of at least a portion of the piston driver being provided with a rack gear.

If the activation member is a wheel member, the wheel member may be arranged in the handle portion such that only a segment of the wheel periphery is accessible for operation by the user at any time. For example, a slot in the handle portion may be provided through which a segment of the wheel member protrudes. In line with the above, the length of the slot then determines the maximum angular displacement of the wheel member inducible by the user during a single operation of the wheel member with e.g. a finger. A wheel member thus arranged indicates that the device is capable of being operated with one hand, i.e. the user may intuitively operate the device in the intended simple manner.

In some embodiments of the invention less than 50% of the wheel perimeter is accessible for user operation at any time.

In some embodiments of the invention less than 30% of the wheel perimeter is accessible for user operation at any time.

The activation member may alternatively be e.g. a part of a wheel, a knob member, a lever, a belt, a touch surface etc.

The activation member may be arranged such that the user operable segment is accessible from the proximal end and/or from a side of the handle portion and such that it is operable in both directions about the axis of rotation.

By requiring a rotary element to be operated for piston activation instead of e.g. a push button the risk of the user accidentally applying an overly large driving force to the piston driver is significantly reduced. The rotary element arrangement provides the user with a much greater motion control, so if the piston initially sticks to the container wall the extra force the user has to apply to overcome the static friction forces will not risk resulting in the piston suddenly being driven a substantial distance through the container at a high speed. Further, by limiting the possible extent of rotation of the activation member during a single operation, it is ensured that should the piston suddenly slip the container wall when a large rotary force is applied then the axial distance which the piston can travel as a result thereof is limited to a fraction of the total possible travel length of the piston.

In another aspect of the invention a drug mixing and delivery device is provided comprising a container comprising an outlet portion and a proximal end portion and extending along a first axis, a first piston arranged in the container between the outlet portion and the proximal end portion, and a second piston arranged in the container between the first piston and the proximal end portion. Thereby, a first chamber is provided between the outlet portion and the first piston and a second chamber is provided between the first piston and the second piston. In a non-use situation the outlet portion may be closed by an appropriate openable, removable and/or penetrable closure part. The first chamber is adapted to initially store a first substance, e.g. a powdered drug or a liquid, and the second chamber is adapted to initially store a second substance, e.g. a liquid. A passage arrangement is provided for enabling fluid passage from the second chamber to the first chamber when the device is in a mixing state. This passage arrangement may e.g. comprise a radial enlargement of the container wall along a certain length of the container which is slightly longer than the axial extent of the first piston so as to thereby provide a bypass channel. The device further comprises a piston driver adapted to interact with the second piston, a handle portion, and an activation member supported by the handle portion. The activation member is operatively coupled with the piston driver and arranged rotatably about a second axis perpendicular to the first axis. At least a portion of the activation member is accessible from the exterior of the handle portion to enable user selective movement of the second piston in the container. Thereby, a user of the device will be able to operate it with one hand, e.g. by holding the handle portion in the palm of the hand while rotating the activation member about the second axis with the thumb.

In use, such a device undergoes a number of different stages, for example a mixing stage during which the second substance is moved from the second chamber to the first chamber (e.g. by collapsing of the second chamber) to mix with the first substance, a priming stage during which the first chamber is de-aerated, an aspiration stage during which the pistons are retracted in the container following insertion of a delivery element into the body of a subject to thereby aspirate a volume of body fluid into the device and enable a visual inspection of which compartment the delivery element is positioned in, and an infusion stage during which the mixed product is actually administered to the subject.

During said mixing stage it is crucial that the second chamber is not collapsed too rapidly because a rapid transfer of the second substance to the first chamber is likely to cause an undesired foaming of the final mixed product. In a mixing device where piston advancement is prompted by depression of a push button there is a significant risk of collapsing the second chamber too rapidly, especially when taking the device into use after longer term storage, because of the aforementioned stick-slip phenomenon.

The incorporation of an activation member which is operated by rotation about an axis perpendicular to the primary container axis, or the axis along which the pistons move, reduces this risk significantly because the operator has a much better control of a rotary activation member movement than of a push button movement and thereby a much better control of the force profile applied to the piston driver. Notably without having to compromise the one handed operation.

The second piston may be adapted to non-releasably couple with the first piston at the completion of transfer of the second substance to the first chamber. In that respect, the second piston may e.g. be provided with a coupling member for interlocking to the first piston immediately following a collapse of the second chamber. This ensures that all subsequent movements of the two pistons are mutual.

The activation member may be adapted to be rotated in a first direction to cause distal, or forward, movement of the second piston in the container and further to be rotated in a second, opposite direction to cause proximal, or backward, movement of the second piston in the container. Backward movement is relevant in relation to the aspiration stage after mixing of the substances and de-aeration of the first chamber. A non-releasable coupling of the two pistons, e.g. as described above, will ensure a mutual retraction thereof and thereby that a proper vacuum can be created in the container for reliable suction of body fluid from the compartment where the delivery element is inserted.

Aspiration is performed to make sure that e.g. an infusion needle is positioned correctly in the body. In some cases the medicament must be administered intravenously and it is therefore important to check that the infusion needle has in fact been inserted into a vein. By creating a vacuum in the container body fluid in the immediate vicinity of the infusion needle is aspirated and the user can verify if the fluid in the container becomes red, an indication that a blood vessel has been penetrated. In practice, it is not only relevant to perform an aspiration before the beginning of the infusion stage. It may be just as relevant during the infusion if doubt arises as to whether the infusion needle may have become dislodged, e.g. due to a sudden movement of the body part receiving the drug. The rotary arrangement of the activation member makes it easy to shift between infusion and aspiration because the user simply has to reverse his/her operation of the activation member and this can be done without changing the position of the hand on the handle portion.

In particular embodiments of the invention the activation member is a wheel member as described above in relation to the first aspect of the invention. The wheel member is coupled with the piston driver such that the direction of motion of the part of the user that operates the wheel member equals the direction of motion of the portion of the piston driver which interacts with the piston. This entails an intuitive handling procedure because the user will expect the wheel to be able to rotate in both directions and will be able to relate the respective directions to the resulting movement of the piston(s). In other words, the user will intuitively understand that a rotation of the operable wheel segment towards the outlet results in a distal, or forward, movement of the piston(s) and that an opposite rotation results in a proximal, or backward, movement of the piston(s).

The wheel member may be arranged in a medial, or centre, plane of the drug delivery device, e.g. such that a portion of it protrudes from the middle section of one handle portion side and/or of the handle portion end surface. Thereby, an operationally symmetrical device is provided which a user can operate with equal ease regardless of which hand is used.

Alternatively, the wheel member may be arranged laterally of the medial plane, whereby the device can be tailor made to suit either left handed or right handed people. A lateral arrangement of the wheel member may require fewer constructional components than a medial arrangement, as will be clear from the below.

The piston driver may be adapted to interact with the second piston directly or via a separate piston pusher. The piston driver may be rigid, flexible or partly flexible in the sense that portions of the piston driver are rigid while others are flexible. The piston driver may e.g. comprise a number of rigid segments hinged edge to edge, thereby providing a longitudinally rigid but transversally deflectable construction. A flexible, or partly flexible, piston driver enables a shorter, more compact drug delivery device, which is desirable in some situations.

A flexible, or partly flexible, piston driver may be guided by a dedicated geometry, such as a groove, in the device, e.g. in the handle portion. The geometry may be designed to promote a non-linear configuration of the piston driver and may assist in ensuring an operational coupling with the activation member. A rack may be provided along at least a portion of the piston driver for engagement with the activation member or an interface member, e.g. a gear, coupled therewith. In particular embodiments, the piston driver is bendable about the second axis.

In case the activation member is a wheel member arranged in a medial plane of the device a two-piece piston driver may be incorporated to enable a symmetrical force distribution on the second piston. A symmetrical force distribution reduces the total axial force needed for piston movement and further eliminates a potential tilting of the second piston in the container. The two piston driver pieces may be arranged in parallel on either side of the wheel member and may each be in engagement with a pinion gear on the wheel member to provide for a transfer of wheel member movements to the second piston.

In case the activation member is a wheel member arranged laterally of the medial plane a single-piece piston driver is sufficient to provide an axisymmetrical force distribution to the second piston, since a piston driver positioned on the medial side of the wheel member can be arranged to move along the centre axis of the container.

The second, or rear, chamber between the two pistons may be filled with a liquid before the first use of the device. In that case, if the device is exposed to fluctuations in the temperature of its surroundings the pistons may displace somewhat from their initial positions. For example, if the device is exposed to frost the liquid in the second chamber may expand and exert repelling forces on the two pistons. In a device having a pre-mounted piston driver the piston drive system will normally resist proximal, or backward, motion of the operated rear piston, whereby the entire expansion of the liquid will be accommodated by displacement of the front piston. However, in this case, to enable a short device the first, or front, piston may initially be arranged close to the bypass channel, so the extent of a premature distal travel of the front piston should be minimised as it may otherwise place the piston in the bypass channel, entailing an uncontrollable and potentially premature subsequent mixing of the two substances.

An initial clearance between the piston driver and the second, or rear, piston may be provided to allow for free proximal displacement of the rear piston before use of the device in response to e.g. forces from an expanding liquid. Thereby, the expansion of the liquid may be accommodated by a displacement of both pistons, in opposite directions, which will reduce the risk of the front piston being prematurely displaced to the bypass channel. In that case when taking the device into use the operator first has to manipulate the activation member to advance the piston driver till it engages with the rear piston, or with an intermediate member coupled with the rear piston. Only from then on will a manipulation of the activation member affect the rear piston.

Alternatively, the front piston may initially be arranged sufficiently far from the bypass channel to eliminate the possibility of it becoming prematurely displaced into the bypass channel. In that case the piston driver may be coupled with the rear piston when the device is supplied from the manufacturer.

Once the substances have been mixed and the device has been de-aerated the front portion of the front piston will be positioned distally of the bypass channel. When the user decides to perform an aspiration the activation member is operated to retract the piston driver, whereby the pistons will be retracted as well. It is, however, desirable to ensure that the pistons cannot by accident be retracted to a point where the front portion of the front piston is once again positioned within the bypass section since this will result in a small volume of the final mixed product being able to fill the bypass channel. Such a volume is potentially wasted and as a consequence the deliverable dose will be smaller than the intended dose. Furthermore, the final mixed product may be so expensive that it becomes quite costly if even a small volume is wasted.

The device may therefore be provided with a non-return mechanism which prevents the front piston from being retracted beyond a position where at least a portion of it covers the distal most portion of the bypass channel.

The non-return mechanism may e.g. comprise an engagement element, such as a pawl member, adapted to engage a portion of the piston driver to prevent retraction of the latter beyond a certain point once the piston driver has passed this point in a piston advancing motion. The engagement element may be associated with the handle portion, e.g. as an integrated flexible arm of an inner casing portion or as a separate element fixedly attached to the handle portion, and the piston driver may comprise a protrusion suitable for being engaged by the engagement element.

In yet another aspect of the invention a method for administering a volume of a drug is provided, the method comprising rotating an operable member (e.g. a wheel element or a disc element) in a first direction about an axis of rotation, the operable member being coupled with an actuator (e.g. via a rack and pinion interface) of which at least a portion in response thereto performs a movement along a general axis perpendicular to the axis of rotation towards an outlet of a reservoir comprising the drug, thereby causing a movable wall member in the reservoir to decrease the volume of the reservoir.

The method may further comprise rotating the operable member in the opposite direction, whereby at least a portion of the actuator in response performs a movement along the general axis away from the outlet, thereby causing the movable wall member to increase the volume of the reservoir.

A further aspect of the invention concerns a use of a wheel element in a drug delivery device for causing movement of at least one piston along a first axis, the wheel element being rotatable about a second axis perpendicular to the first axis. The at least one piston may e.g. be caused to move in a drug container from an initial position to a position where at least a volume of drug has been expelled from the container.

In particular embodiments the wheel element is used as an activation button in the drug delivery device for effecting the piston motion, the use comprising rotating the wheel element about its axis of rotation, thereby activating a piston driver to move a piston along an axis perpendicular to the axis of rotation. Less than the entire perimeter of the wheel element may be accessible for user operation at any time.

The use may further comprise rotating the wheel element in a first direction to activate the piston driver to move the piston in a first direction along the axis perpendicular to the axis of rotation and rotating the wheel element in a second direction opposite to the first direction to activate the piston driver to reverse the movement of the piston.

The various types of drug delivery devices described in the present text may be purely mechanical, i.e. devoid of any electronics, or they may be electro-mechanical. A purely mechanical device is generally less expensive to produce than a device which carries electronic parts and is therefore normally more attractive vis-à-vis a disposable medical device segment. Further, the mixing devices may be integrated devices, i.e. they may comprise, individually, all necessary features to carry out substance mixing and final product delivery without use of external parts or devices.

The substances to be mixed may e.g. be two liquids or a dry powder and a liquid, such as e.g. a freeze-dried drug and a solvent. The final product may be adapted to be delivered in a single dose, e.g. the container may be emptied or substantially emptied in one go after the mixing of the substances, or in multiple doses.

It will be clear to a person skilled in the art that the term “perpendicular”, in the present context, not only covers perpendicular in the strict mathematical sense but also “substantially perpendicular”, accommodating any slight deviation from right angles that may occur in practice within mechanical construction.

Further, in the present context, the term “user operable” should be understood as “having an interface for user manipulation”, i.e. a user operable element is an element of which at least a portion is accessible for contact and direct operation by a subject.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of features, structures and/or characteristics described in relation to the invention is encompassed by the invention unless expressly stated herein or clearly contradicted by context.

The use of any and all examples, or exemplary language (e.g., such as, etc.), in the text is intended merely to illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a perspective top view of a drug mixing and delivery device according to a first embodiment of the invention,

FIG. 2 is a perspective exploded view of the device of FIG. 1,

FIG. 3 is a perspective bottom view of the device of FIG. 1, where the bottom shell of the handle portion has been removed to provide a view to the inner construction,

FIG. 4 is a top perspective view of a drug mixing and delivery device according to a second embodiment of the invention,

FIG. 5 is a perspective exploded view of the device of FIG. 4,

FIG. 6 is a perspective view of an exemplary piston driver applicable in the device of FIG. 4,

FIG. 7 is a perspective view of an exemplary piston drive mechanism applicable in the device of FIG. 4,

FIGS. 8a-d show an operating sequence for the device of FIG. 4,

FIG. 9 is a close-up perspective view of an exemplary non-return mechanism,

FIG. 10 is a perspective view of the handle portion of a drug mixing and delivery device according to a third embodiment of the invention,

FIG. 11 is a detailed view of an activation wheel and a piston rod segment applicable in the device of FIG. 10,

FIG. 12 shows an interaction between the wheel and the piston rod segment of FIG. 11, and

FIGS. 13-16 show different types of drug delivery devices according to alternative embodiments of the invention.

In the figures like structures are mainly identified by like reference numerals.

### DESCRIPTION OF EXEMPLARY EMBODIMENTS

When in the following relative expressions, such as “clockwise” and “counter-clockwise” and “right” and “left”, are used, these refer to the appended figures and not necessarily to an actual situation of use. The shown figures are schematic representations for which reason the configuration of the different structures as well as their relative dimensions are intended to serve illustrative purposes only.

An exemplary drug delivery device embodying the principles of the present invention comprises a reservoir with a piston, which piston is movable along a general axis, and an activation element adapted to cause movement of the piston in cooperation with a piston drive member, which activation element is rotatable about an axis perpendicular to the general axis.

With such a device the user has greater control of the force profile applied to the piston than users of conventional prior art devices. Furthermore, the rotatable activation element as arranged in accordance with embodiments of the invention requires a repetitive operational motion in order to cause the piston to move more than a fraction of the total intended travel length in the container. This gives the user a good control of the motion profile of the piston in the reservoir.

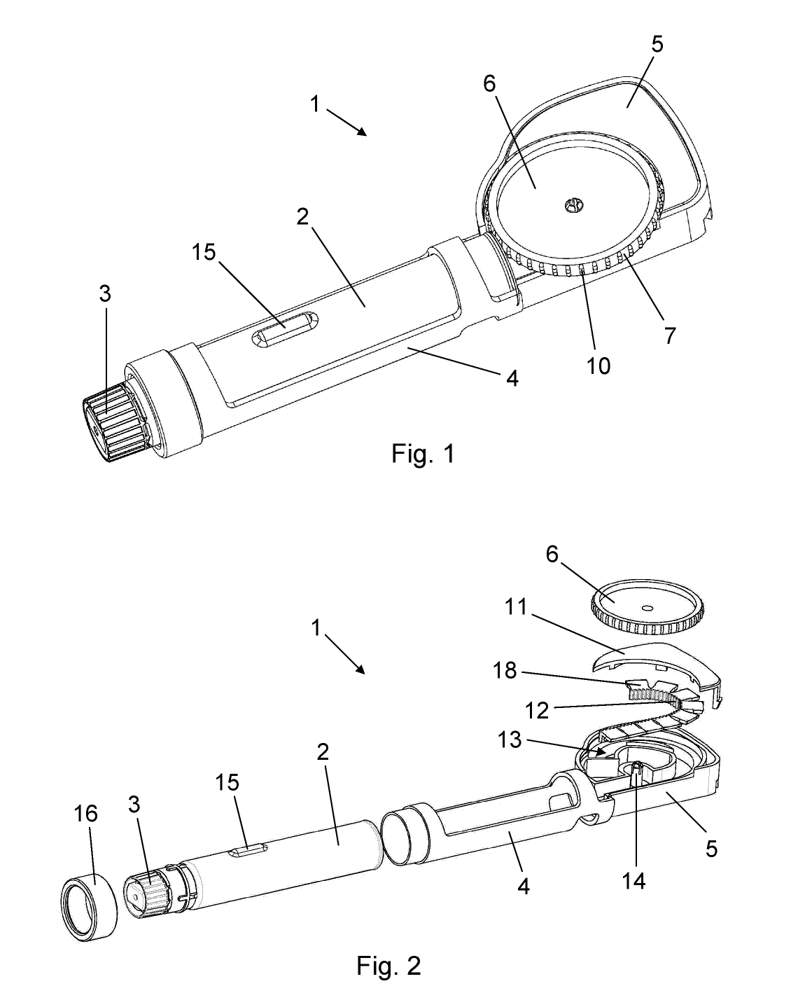

FIG. 1 shows, in a first embodiment of the invention, a drug mixing device 1 which comprises a cartridge holder 4 for a dual chamber cartridge 2, e.g. of glass or plastic, holding an amount of powdered medicament (not visible) in a front chamber and a volume of diluent (not visible) in a rear chamber. The two chambers are separated by a front piston (not visible) which in the pre-activation state of the mixing device 1 is positioned proximally of a bypass channel 15 shaped as a groove in the cartridge wall. A closure 3 is provided at the outlet end of the cartridge 2 for user selective opening thereof and further a rear piston (not visible) seals the end portion of the cartridge 2 opposite the outlet end. The mixing device 1 further comprises a handle 5 being either attached to or integral with the cartridge holder 4. The handle 5 houses a thumb wheel 6 which is mounted so as to enable user operation thereof by manipulation of a free wheel periphery portion 7. The thumb wheel 6 has along its periphery a number of circumferentially spaced apart small protrusions 10 which provide for a good operational contact interface with a finger.

FIG. 2 is an exploded view of the mixing device 1 showing the constructional components thereof. The handle 5 houses, apart from the thumb wheel 6, a piston rod 12 which consists of a number of piston rod segments 18 integrally hinged along respective edge portions. This configuration provides a transverse flexibility, which allows the piston rod 12 to bend about an axis perpendicular to its own length axis, as well as an axial rigidity. The piston rod 12 is arranged in a curved track 13 that serves both as a storage compartment and a guide for the movements of the piston rod 12. The thumb wheel 6 is arranged rotatably on a shaft 14. A top plate 11 is also provided as a part of the handle 5. A locking ring 16 is fitted over the distal end portion of the cartridge holder 4 to keep the cartridge 2 in place in the cartridge holder 4.

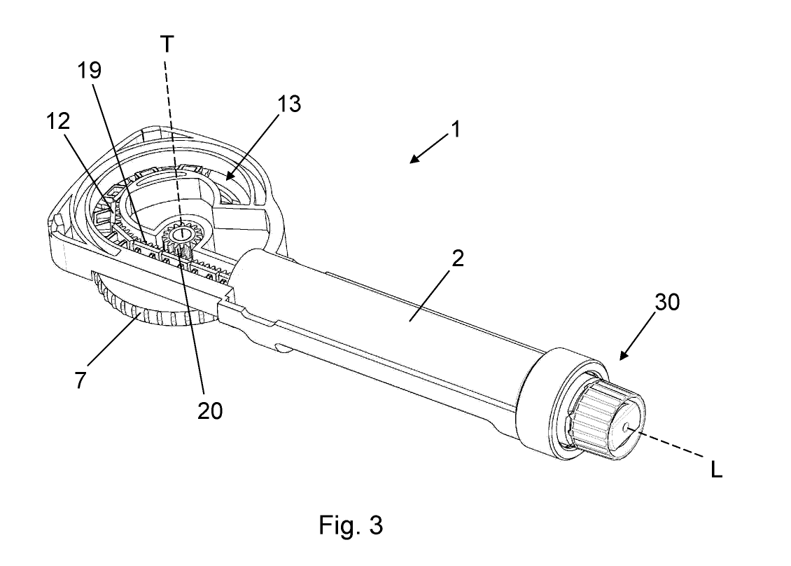

FIG. 3 shows a perspective bottom view of the mixing device 1 where the bottom plate has been removed to show the interaction of the internal components. Here, the piston rod 12 is seen in the curved track 13. The piston rod 12 is provided with a rack gear 19 along a substantial portion of its length. This rack gear 19 is in mesh with a pinion gear 20 on the underside of the thumb wheel 6 such that if the thumb wheel 6 is rotated counter-clockwise about a transversal axis of rotation T, by movement of the free wheel periphery portion 7 towards a distal end portion 30, the pinion gear 20 will rotate counter-clockwise as well and thereby cause a forward movement of the piston rod 12 in the track 13. The portion of the piston rod 12 which is positioned distally of the pinion gear 20 will as a consequence perform a distal movement along a longitudinal axis L, which will in turn activate the rear piston (not visible) in the cartridge 2. Conversely, a clockwise rotation of the thumb wheel 6 will cause a clockwise rotation of the pinion gear 20 and thereby a reverse, or backward, movement of the piston rod 12. The thumb wheel 6 is arranged in the handle 5 such that, at any time, a diameter of the thumb wheel is substantially parallel with the longitudinal axis L of the cartridge 2, i.e. the transversal axis of rotation T and the longitudinal axis L are, at least substantially, at right angles.

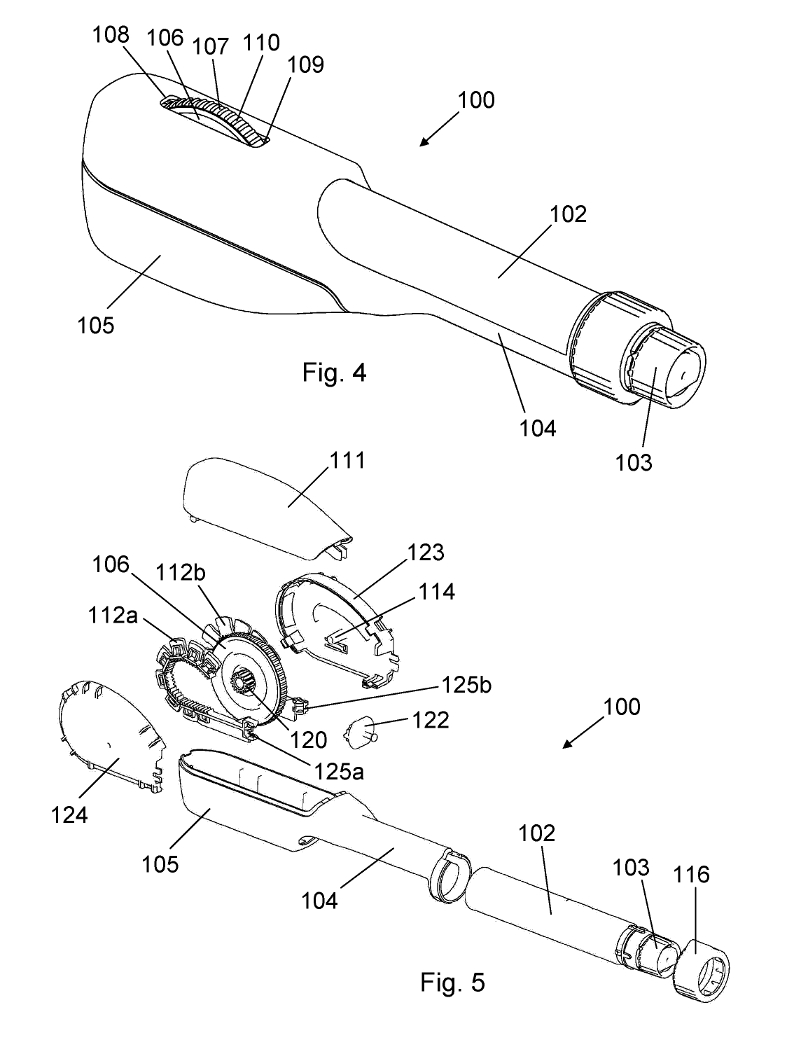

FIG. 4 shows a perspective top view of a mixing device 100 according to another embodiment of the invention. Similar to the mixing device 1 of the previous embodiment the mixing device 100 comprises a cartridge holder 104 holding a dual chamber cartridge 102 having a user operable closure 103 and comprising a front piston (not visible) separating the two chambers and a rear piston (not visible) sealing the proximal end portion of the cartridge 102. The mixing device 100 further comprises a handle 105 housing a thumb wheel 106 with a number of circumferentially spaced apart protrusions 110 distributed along its periphery. In contrast, however, to the mixing device 1 of the previous embodiment the handle 105 is provided with a slot through which a free wheel periphery portion 107 protrudes. The slot extends along the longitudinal axis of the handle 105 between a proximal edge 108 and a distal edge 109. This incites the user to operate the thumb wheel 106 in a familiar fashion like a scroll wheel on a computer mouse. The distance between the proximal edge 108 and the distal edge 109 determines the maximum angular displacement of the thumb wheel 106 during one finger manipulation of the free wheel periphery portion 107.

FIG. 5 shows an exploded bottom view of the mixing device 100 detailing the interior components. The handle 105 and the cartridge holder 104 are shaped, e.g. moulded, in one piece and the handle 105 is closed by a bottom plate 111. A hollow compartment defined by two inner casing shells 123, 124 is embedded in the handle 105. One inner casing shell 123 carries a shaft 114 on which the thumb wheel 106 is arranged. The mixing device 100 is symmetrical about the longitudinal centre axis of the cartridge 102, i.e. the extension of the centre axis of the cartridge 102 lies in the median plane of the thumb wheel 106. In order to enable a symmetric load distribution on the rear piston a two-piece piston rod 112a, 112b is employed. The piston rod pieces 112a, 112b are arranged on either side of the thumb wheel 106 and each piece is in mesh with a pinion gear 120 on the thumb wheel 106 for transmission of movements between the thumb wheel 106 and the piston rod pieces 112a, 112b. The distal ends of the piston rod pieces 112a, 112b are shaped as claws 125a, 125b adapted to engage with a piston pusher 122 which is capable of connecting to the rear piston in the cartridge 102. The piston rod pieces 112a, 112b both comprise a number of integrally hinged segments providing two transversally flexible structures. The hollow compartment formed by the inner casing shells 123, 124 serves as a storage compartment and a guide for the piston rod pieces 112a, 112b. A locking ring 116 ensures that the cartridge 102 stays in place in the cartridge holder 104.

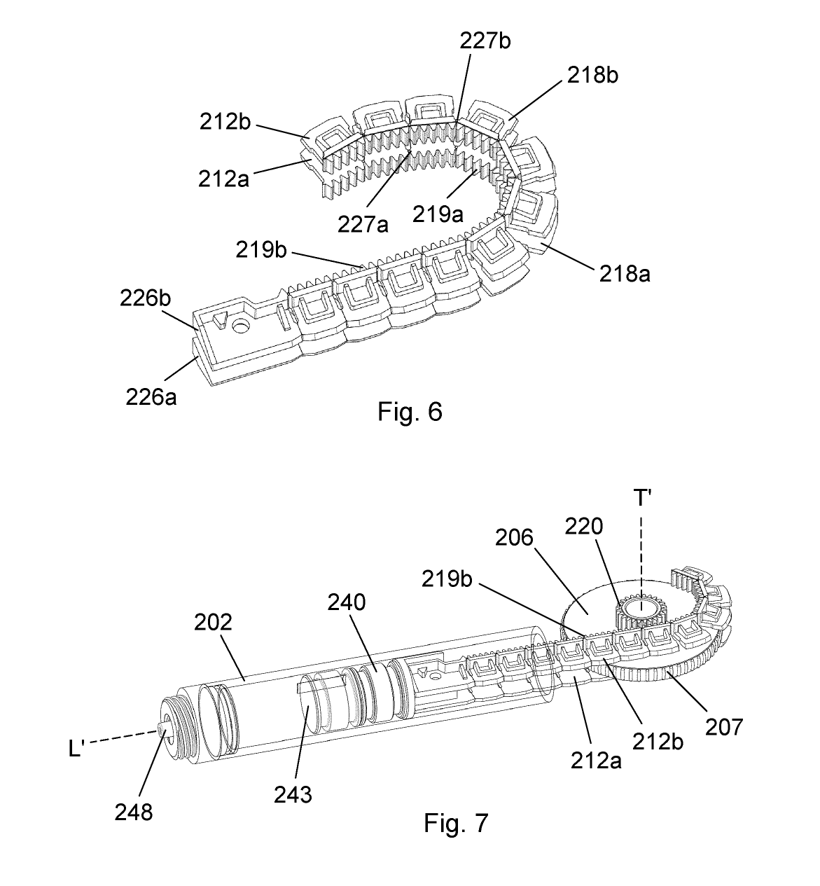

FIG. 6 shows a close up of an exemplary piston rod useable in a mixing device according to an embodiment of the invention. The piston rod comprises two pieces 212a, 212b arranged in parallel and spaced from each other a distance, e.g. corresponding to the thickness of the thumb wheel 106 previously disclosed. Each piece 212a, 212b comprises a number of segments 218a, 218b which are connected through integral hinges 227a, 227b to provide a longitudinally rigid but transversally deflectable construction. At the distal ends the pieces 212a, 212b are provided with push members 226a, 226b for cooperation with a piston. Further, each segment 218a, 218b holds a portion of a rack gear 219a, 219b which due to the integral construction of the two pieces 212a, 212b forms part of a continuous rack gear running along substantially the entire length of the piston rod.

FIG. 7 shows the piston rod of FIG. 6 as part of an activation unit of a drug mixing and delivery device according to an embodiment of the invention. The piston rod pieces 212a, 212b are arranged on either side of a thumb wheel 206 and the rack gears 219a, 219b are in mesh with respective pinion gears 220 (only one is visible) on the thumb wheel 206. The distal end portions of the piston rod pieces 212a, 212b are occupied in a cartridge barrel 202 and coupled with a rear piston 240 and a front piston 243 for mutual advancement thereof. The thumb wheel 206 is rotatable about an axis of rotation T′ which is (at least substantially) perpendicular to a longitudinal axis U of the cartridge barrel 202. Manipulation of a peripheral portion 207 of the thumb wheel 206 in a clockwise direction results in a distal motion of the rear and front pistons 240, 243 along the axis U due to the interaction between the rack gears 219a, 219b and the pinion gears 220. In the figure the position of the two pistons correspond to a mixing phase having been completed and a further distal motion thereof will cause first air present in the cartridge barrel 202 and subsequently the mixed product to be expelled through a Luer connection outlet 248.

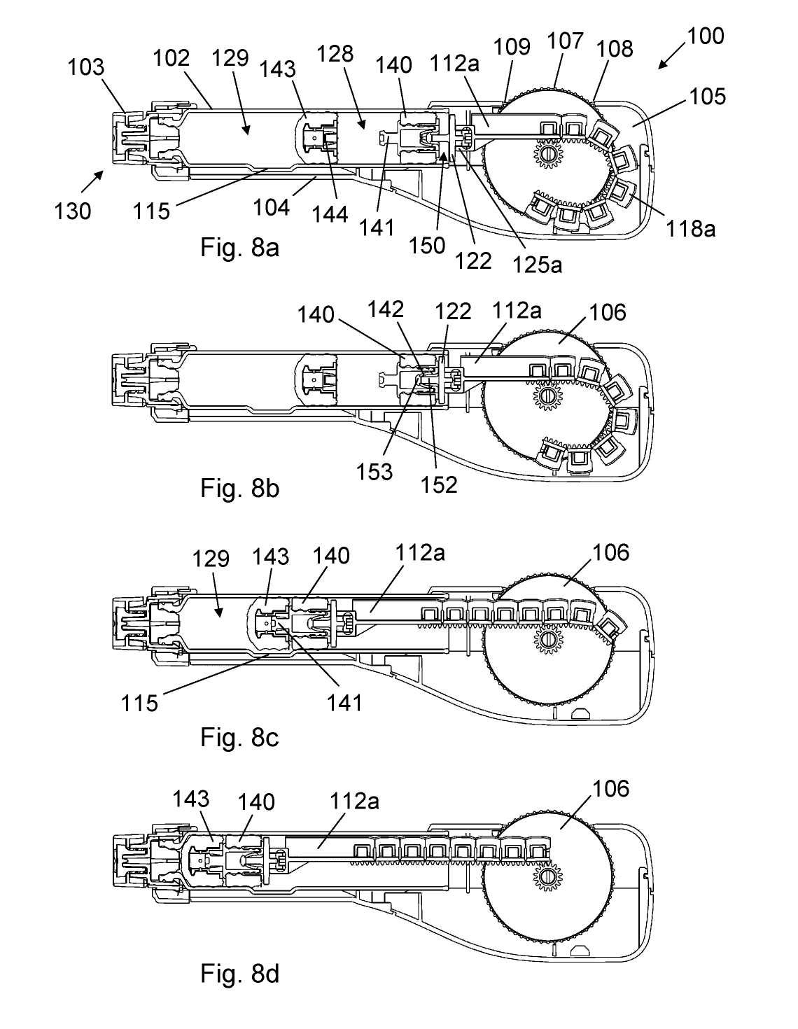

FIGS. 8a-8d are cross-sectional views showing an operation sequence of the mixing device 100 which comprises a diluent (not shown) stored in a rear chamber 128 and a lyophilised drug (not shown) stored in a front chamber 129.

FIG. 8a shows the mixing device 100 in a pre-activation state, e.g. as delivered from the manufacturer. The closure 103 has, however, been opened by a user to allow escape of gas from the interior of the cartridge 102. In this state the piston pusher 122 is decoupled from the rear piston 140 and a clearance 150 is provided between the two. The clearance 150 allows a proximal movement of the rear piston 140 in the cartridge 102 during storage, e.g. to accommodate, or partly accommodate, a potential expansion of the diluent in the rear chamber 128. The rear piston 140 has a distally projecting coupling profile 141 for interlocking engagement with a snap structure 144 on the front piston 143.

FIG. 8b shows the mixing device 100 in a ready-to-mix state where the user has executed a small counter-clockwise rotation of the thumb wheel 106 sufficient to drive the piston rod pieces 112a, 112b forward and thereby advance the piston pusher 122 a distance corresponding to the clearance 150. During this advancement a coupling head 153 provided at the end of a neck portion 152 of the piston pusher 122 is forced into engagement with a snap structure 142 of the rear piston 140 to thereby mechanically secure the piston pusher 122 to the rear piston 140. Thus, all subsequent motions of the piston pusher 122 and the rear piston 140 are mutual. The user can now begin the process of transferring the diluent from the rear chamber 128 to the front chamber 129 to reconstitute the powdered drug therein. This is done by further counter-clockwise rotation of the thumb wheel 106, i.e. by repetitive manipulation of the free wheel periphery 107 between the proximal edge 108 and the distal edge 109.

FIG. 8c shows the mixing device 100 in an end-of-reconstitution state where the rear piston 140 has been advanced to collapse the rear chamber 128 and interlock with the front piston 143. During the initial advancement of the rear piston 140 the diluent in the rear chamber becomes pressurised and drives the front piston 143 distally until it takes up a position within the boundaries of the bypass channel 115. At this point continued advancement of the rear piston 140 forces the diluent into the bypass channel 115 and further on into the front chamber 129. When all of the diluent has been thus transferred to the front chamber 129 and the rear chamber 128 is completely collapsed the coupling profile 141 engages with the snap structure 144 to interlock the rear piston 140 and the front piston 143. Thereby, all subsequent motions of the rear piston 140 and the front piston 143 are mutual.

FIG. 8d shows the mixing device 100 in an empty state where the front piston 143 has been pushed all the way to the distal end of the cartridge 102 to expel the entire contents into a subject (not shown) via a conventional infusion set (also not shown) attached to the distal end portion 130 and fluidly connecting the outlet of the cartridge 102 with the body of the subject.

At any point between the end-of-reconstitution state and the empty state it is possible for the user to perform an aspiration to check whether the infusion needle is correctly positioned in the body. The aspiration is performed simply by reversing the operation of the thumb wheel 106, i.e. by rotating the free wheel periphery 107 clockwise instead of counter-clockwise. Since the rear piston 140 and the front piston 143 are interlocked a retraction of the piston rod pieces 112a, 112b will result in a collective retraction of the two pistons in the cartridge 102. The rack and pinion coupling between the thumb wheel 106 and the piston rod pieces 112a, 112b together with the operational interface to the user allows all piston movements to be carried out in a controlled manner without any risk of causing foaming of the reconstituted drug or other undesired effects of a too rapid piston motion.

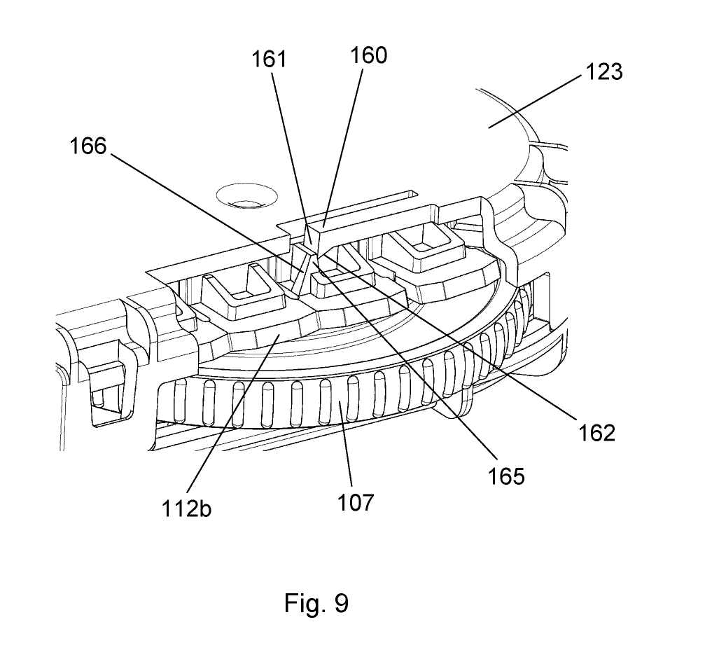

FIG. 9 shows an embodiment of a non-return mechanism which may be incorporated in the mixing device 100 to prevent that the front piston 143, following the reconstitution and a de-aeration of the cartridge 102, becomes retracted back into the boundaries of the bypass channel 115 (for the sake of clarity a section of the inner casing shell 123 has been removed from the figure). In that respect, a small protrusion or wall 165 is provided on a segment of one piston rod piece 112b and a flexible ratchet arm 160 is provided as an integrated portion of the inner casing shell 123. The wall 165 comprises a distal ramp 166 and a proximal abutment face and the ratchet arm 160 comprises a proximal inclined face 162 adapted to ride over the wall 165 along the ramp 166 when the piston rod piece 112b moves distally relative to the inner casing shell 123 and a distal abutment face 161 adapted to abut with the proximal abutment face of the wall 165 to prevent proximal movement of the piston rod piece 112b beyond that point. The exact placement of the wall 165 on the piston rod piece 112b determines the extent of piston retraction. In this particular embodiment the wall 165 is arranged such that once the distal portion of the front piston 143 has been advanced to a position distally of the distal edge of the bypass channel 115 the flexible ratchet arm 160 passes the top of the wall 165 and snaps in behind it to prevent a proximal motion of the distal portion of the piston rod 112a, 112b, and thereby a retraction of the front piston 143, beyond that point. With reference to the above described possibility of performing an aspiration it is noted that when such a non-return mechanism is incorporated the pistons 140, 143 must in practice be advanced a little further distally in the cartridge 102 from their respective positions at the non-return point before aspiration is actually possible.

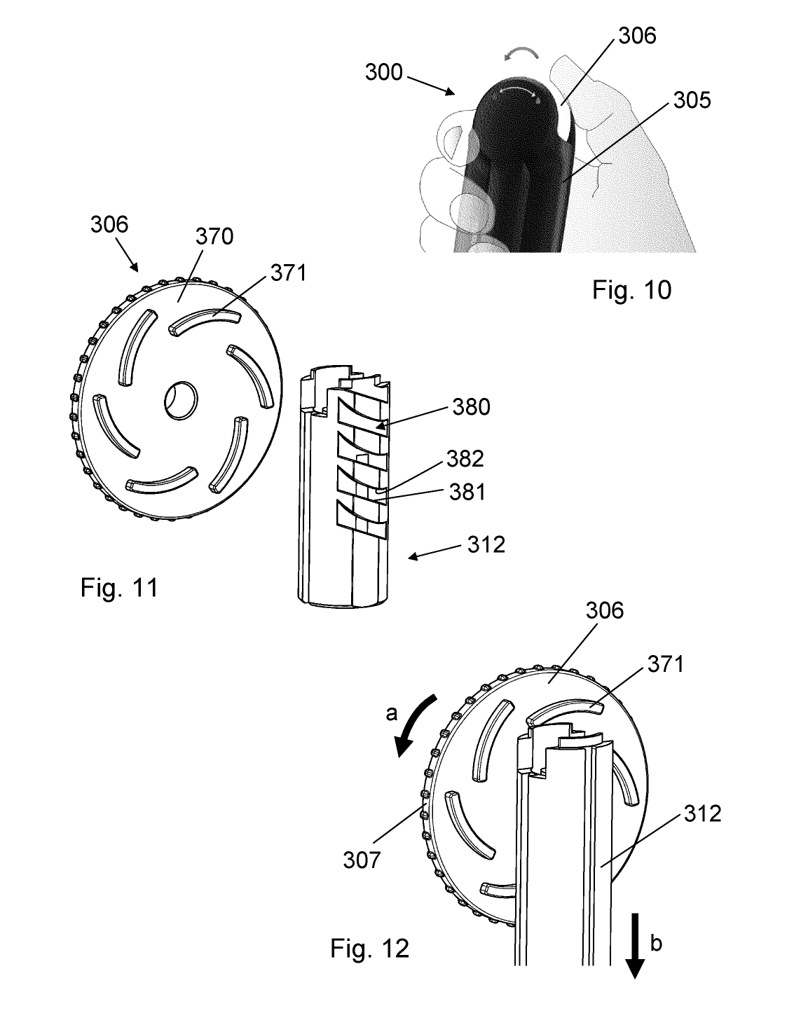

FIG. 10 shows a mixing device 300 according to yet another embodiment of the invention. The mixing device 300 comprises a handle 305 housing a thumb wheel 306 and a telescopic piston rod (not visible), the distal portion of which is coupled with a rear piston (not shown) in a dual chamber cartridge (also not shown). A portion of the periphery of the thumb wheel 306 is available for operation at the proximal end of the handle 305. The thumb wheel 306 and the piston rod are coupled such that a rotation of the thumb wheel 306 in the direction of the arrow results in a retraction of the piston rod and a rotation of the thumb wheel 306 in the direction opposite the arrow results in an advancement of the piston rod.

FIG. 11 shows the coupling interface between the thumb wheel 306 and a proximal portion of the piston rod 312. A number of curved cams 371 are disposed on a wheel side 370. The cams 371 are adapted to successively engage with a respective groove 380 in the proximal portion of the piston rod 312. The grooves each comprise a straight edge 381 and a curved edge 382 adapted to receive a cam 371. The proximal portion of the piston rod 312 is coupled directly to the wheel side 370 along a diameter of the thumb wheel 306, as shown in FIG. 12. By rotation of the thumb wheel 306 in a direction a, via manipulation of a wheel periphery 307, a cam 371 first enters a groove 380, then traverses the groove 380 along the curved edge 382 and finally leaves the groove 380. This relative motion between the cam 371 and the groove 382 results in a displacement of the proximal portion of the piston rod 312 in a direction b. When the cam 371 leaves the groove 380 another cam 371 on the wheel side 370 enters another groove 380 situated proximally of the previously traversed groove 380 and so on, moving the piston rod continuously forward in the direction b. Notably, a reversal of the rotation of the thumb wheel 306 leads to an opposite translational movement of the piston rod. This type of direct coupling of the thumb wheel 306 to the proximal portion of the piston rod 312 spares an intermediate coupling element, such as e.g. a pinion gear.

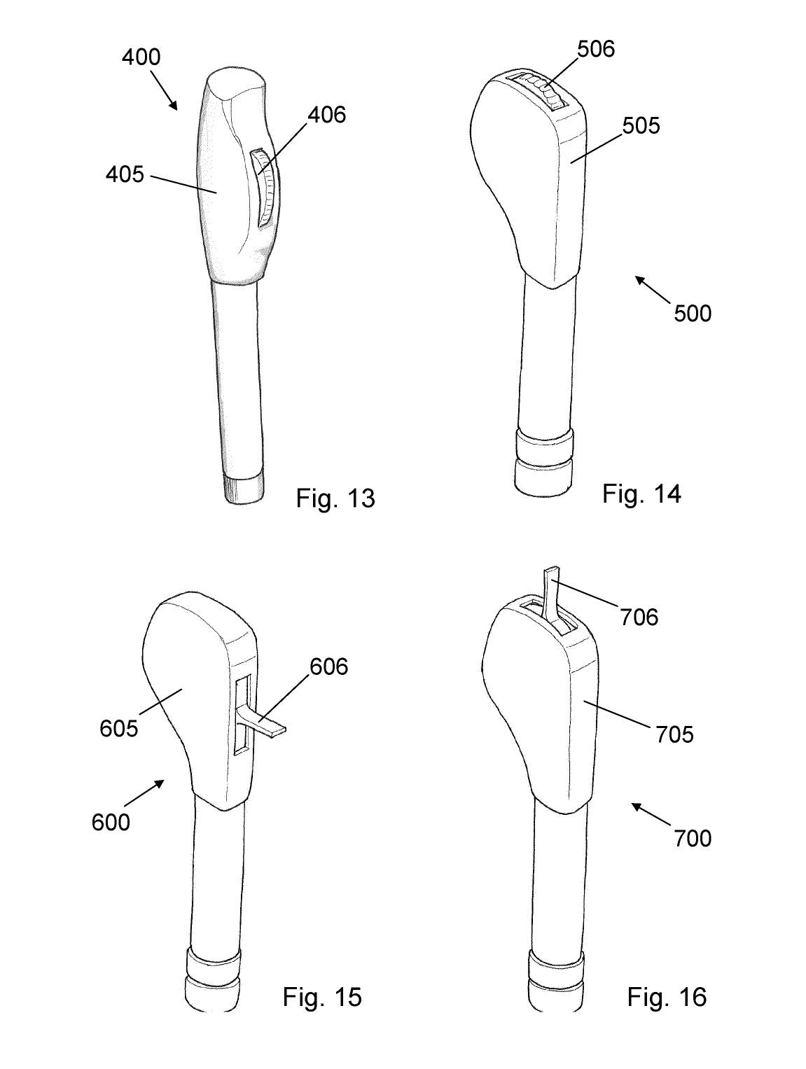

FIG. 13 shows a mixing device 400 according to a further embodiment of the invention. In this embodiment a pear shaped handle 405 houses a linear rigid piston rod (not visible) along with a thumb wheel 406.

FIG. 14 shows a mixing device 500 according to yet another embodiment of the invention. The mixing device 500 structurally resembles the mixing device 100 of FIG. 4, except that a thumb wheel 506 protrudes through an opening in the proximal end face of an operations handle 505.

FIG. 15 shows a mixing device 600 according to yet another embodiment of the invention. The mixing device 600 functionally resembles the mixing device of FIG. 4, and it comprises a handle 605 and a bendable piston rod (not visible). The piston rod is operable via an activetion lever 606 protruding through an opening in a side portion of the handle 605. The activation lever 606 is coupled with the piston rod such that the piston rod is advanced when the activation lever 606 is rocked towards the distal end of the mixing device 600 and retracted when the activation lever 606 is rocked towards the proximal end of the mixing device 600.

FIG. 16 shows a mixing device 700 according to yet another embodiment of the invention. The mixing device 700 comprises a handle 705 and a rockable activation lever 706 protruding through an opening in the proximal end face of the handle 705. A bendable piston rod housed in the handle 705 is operable via the activation lever 706. The activation lever 706 and the piston rod are coupled such that the piston rod is advanced when the activation lever 706 is rocked towards the right and retracted when the activation lever 706 is rocked towards the left.

The invention is further described by the following non-limiting embodiments:

A drug delivery device comprising: a container comprising an outlet portion and a proximal end portion, a first piston arranged in the container between the outlet portion and the proximal end portion, defining a first variable volume chamber, a second piston arranged in the container between the first piston and the proximal end portion, defining a second variable volume chamber, the first and second pistons being moveable relative to the container along a first axis, a first substance arranged in the first variable volume chamber, a second substance arranged in the second variable volume chamber, a bypass arrangement adapted to allow a transfer of the second substance to the first variable volume chamber to mix with the first substance and produce an administrable product, a piston driver adapted to move at least the second piston, and a user operable activation member being operatively coupled with the piston driver and adapted to move the piston driver between an initial position and a position where at least a volume of the administrable product has been expelled through the outlet portion, wherein the activation member is rotatable about a second axis perpendicular to the first axis.

A drug mixing and delivery device comprising: a reservoir comprising a first end portion, a second end portion and a cylindrical wall extending therebetween, the first end portion comprising an outlet, a first movable wall member arranged in the reservoir between the first end portion and the second end portion, a second movable wall member arranged in the reservoir between the first movable wall member and the second end portion, a channel arrangement enabling transfer of fluid from a compartment between the first movable wall and the second movable wall to a compartment between the first movable wall and the first end portion, a drive member adapted to move at least the second movable wall member along a first axis, a handle portion, and a user operable activation element rotatably arranged in the handle portion, wherein the activation element is adapted to rotate about a second axis perpendicular to the first axis.

The activation element may be a disc member or a wheel member, such as a drive wheel or a thumb wheel.

1. A drug delivery device comprising:
a container comprising an outlet portion and a proximal end portion,
a first piston arranged in the container between the outlet portion and the proximal end portion, defining a first variable volume chamber capable of holding a first substance,
a second piston arranged in the container between the first piston and the proximal end portion, defining a second variable volume chamber capable of holding a second substance, the first and second pistons being movable relative to the container along a first axis,
a bypass arrangement adapted to allow fluid transfer from the second variable volume chamber to the first variable volume chamber for provision of an administrable product in the first variable volume chamber,
a piston driver adapted to move at least the second piston,
a user operable activation member operatively coupled with the piston driver and adapted to move the piston driver between an initial position and a position where at least a volume of the administrable product has been expelled through the outlet portion, the activation member being rotatable about a second axis perpendicular to the first axis, and
a handle portion adapted to support the activation member,
wherein the container is arranged stationarily relative to the handle portion.

2. A device according to claim 1, wherein the activation member is rotatable in both a first direction and a second direction, and wherein, following the provision of the administrable product, the piston driver is adapted to move the second piston distally towards the outlet portion in response to a rotation of the activation member in the first direction and to move the second piston proximally away from the outlet portion in response to a rotation of the activation member in the second direction.

3. A device according to claim 1, wherein the handle portion comprises an opening through which the activation member protrudes.

4. A device according to claim 1, wherein the activation member is arranged in a medial plane of the handle portion.

5. A device according to claim 1, wherein the piston driver is bendable about the second axis.

6. A device according to claim 1, wherein the activation member is a drive wheel, and wherein the piston driver comprises two parallel piston rods, arranged on either side of the drive wheel.

7. A device according to claim 1, wherein the activation member is arranged laterally of a medial plane of the handle portion.

8. A device according to claim 1, wherein the activation member is a drive wheel comprising engagement segments distributed on a planar surface thereof and adapted to successively engage with respective complementary engagement segments on the piston driver.

9. A device according to claim 1, wherein the first piston and the second piston are adapted to interlock upon a complete collapse of the second variable volume chamber.

10. A device according to claim 1, wherein, in a pre-activated state of the device, a clearance is provided between the piston driver and the second piston.

11. A device according to claim 1, further comprising an engagement element associated with the handle portion, the engagement element being adapted to engage with the piston driver to thereby limit the proximal motion of the distal portion of the piston driver.
